(19) United States
(12) Patent Application Publication
(10) Pub. No.: US 2008/0275337 A1
(43) Pub. Date: Nov. 6, 2008

(54) BREATHING DETECTION APPARATUS AND METHOD

(76) Inventors: Helge Fossan, Stavanger (NO); Ken Morallee, Orpington (GB)

Correspondence Address: Karen Lenaburg, Esq.;DORSEY & WHITNEY LLP, Suite 3400, 1420 Fifth Avenue, Seattle, WA, 98101, US

(21) Appl. No.: 12/110,152

(22) Filed: Apr. 25, 2008

Related U.S. Application Data
(60) Provisional application No. 60/915,388, filed on May 1, 2007.

(30) Foreign Application Priority Data
May 8, 2007 (GB) 0708851.1

(51) Int. Cl. A61B 5/055

(52) U.S. Cl. 600/428; 600/430

(57) ABSTRACT

A device is disclosed for remote monitoring of breathing movements of a patient, comprising a sender transmitting a microwave signal toward the patient, a receiver arranged to receive a signal reflected by the patient, and a processor. The processor mixes the received signal with the transmitted signal and with a signal in quadrature with the transmitted signal to determine a breathing frequency of the patient. In some embodiments, two signals at different frequencies are emitted toward the patient and processed in order to compensate for breathing patterns that are not uniquely resolvable at a single frequency.

### CROSS-REFERENCE TO RELATED APPLICATIONS

This application claims the benefit of United Kingdom Application No. 0708851.1, filed May 8, 2007, and U.S. Provisional Application No. 60/915,388, filed May 1, 2007. The entire disclosure of these prior applications are considered to be part of the disclosure of the instant application and is hereby incorporated by reference therein.

### TECHNICAL FIELD

This invention relates to systems and methods for monitoring cyclic movements, especially breathing movements of patients.

### BACKGROUND OF THE INVENTION

In a number of different situations there is a need for monitoring breathing movements. For example, in the field of medicine, the recording of breathing volumes and rates in patients is often critical. The recording of breathing volumes and rates in patients is currently either performed by connecting a volume flow-sensing device to the patient's airway or by measuring the mechanical excursions of the chest and abdominal walls. For long-term monitoring purposes, the airway-based techniques are inappropriate since they interfere with normal breathing and are unpleasant for the patient. Although airway-based techniques are currently used in patient's dependent on respiratory-assist devices there may be less intrusive and more reliable means of obtaining such data.

Techniques for breathing monitoring based on chest and abdominal wall movements are either strain gauge based (recording of changes in body circumference length), or based on elastic inductive electrical conductor loops arranged around the chest and abdomen of the patient. Recordings of the inductance of the loops can then be used to estimate the magnitude of cross-sectional area variations of the chest and abdominal compartments. U.S. Pat. No. 4,308,872 and U.S. Pat. No. 6,374,667 show examples of these techniques. These systems require that uncomfortable equipment is wrapped around the patient body.

Microwave and radar systems have also been suggested for monitoring movements. This solution has the advantage of eliminating the need for equipment in contact with the patient, thus reducing the disturbances to a minimum. In European Patent No. 0064788, a number of Doppler radars are used to observe and quantify body movements. The radar beams are aimed at the body from a number of directions to obtain a full coverage of the body movements. A problem related to this device is “phase wrapping” which occurs when the movement becomes large relative to the wavelength of the transmitted radar signal. When the amplitude of the movement exceeds half the wavelength the system is unable to decide whether the movement has shifted back to zero or has continued.

U.S. Pat. No. 4,513,748 describes a system for monitoring heart rhythm, in which one frequency is used to measure breathing movements and another is used to measure heart movements. This document does not describe measurements of chest movements or breathing as it is only aimed at filtering out breathing movements from signals to obtain heart rhythm signals.

U.S. Pat. No. 4,958,638 describes a non-contact vital signs monitor by means of a frequency modulated continuous wave radio signal. According to this patent, the radio signal reflected from the patient is mixed with a transmitted signal to obtain phase information. Phase quadrature signals and frequency modulation are used to be able to handle movements in a range exceeding the wavelength of the carrier frequency. This, however, decreases the resolution and thus the monitor's sensitivity to smaller movements.

### SUMMARY OF THE INVENTION

These and other objects are achieved by a device for remote monitoring of breathing movements of a patient comprising a transmitter arranged to transmit a microwave signal toward the patient, a receiver arranged to receive a signal reflected by the patient, and a processor comprising a first mixer arranged to mix the transmitted signal and the received signal to provide a first signal component, a second mixer arranged to mix the received signal with a signal in quadrature with the transmitted signal to provide a second signal component, and an output device arranged to provide a signal representative of the patient's breathing movements based on the first and second signal components.

The second mixer in the device according to the invention is arranged to mix a signal in quadrature with the transmitted signal (that is with a phase shift of 90 degrees compared with the transmitted signal) and the received signal.

A combination (e.g. the arithmetic sum of the FFT) of the first component and the second component will result in a signal with a first harmonic in the same frequency range as the breathing signal, as long as the object's movement has an amplitude which is smaller than the wavelength. The device according to the invention comprises a Doppler radar that is sensitive to on axis movement with respect to the transducer.

### BRIEF DESCRIPTION OF THE DRAWINGS

FIG. 1 is a block diagram of a device for measuring breathing rates in accordance with an embodiment of the invention.

FIGS. 2A-2D are graphs of signals processed to determine a breathing rate in accordance with an embodiment of the invention.

FIG. 3 is a block diagram of an alternative embodiment of a device for measuring breathing rates using two microwave signals in accordance with an embodiment of the present invention.

FIG. 4 is a graph of signals used to measure breathing rates using two microwave signals in accordance with an embodiment of the present invention.

FIG. 5 is a schematic block diagram of an amplifier for use in a device in accordance with an embodiment of the invention.

FIG. 6 is a digraph illustrating mathematical analysis of the signal processing performed in accordance with an embodiment of the invention.

FIG. 7 is a block diagram of a system for measuring breathing rates in accordance with an embodiment of the present invention.

FIG. 8 is a waveform measured by the system of FIG. 7.

FIG. 9 is a plot of measure rate vs. tone rate for measurements performed with a system in accordance with an embodiment of the present invention.

FIG. 10 is a Bland Altman plot of measurements formed with a system in accordance with an embodiment of the present invention.

### DETAILED DESCRIPTION OF PREFERRED EMBODIMENTS

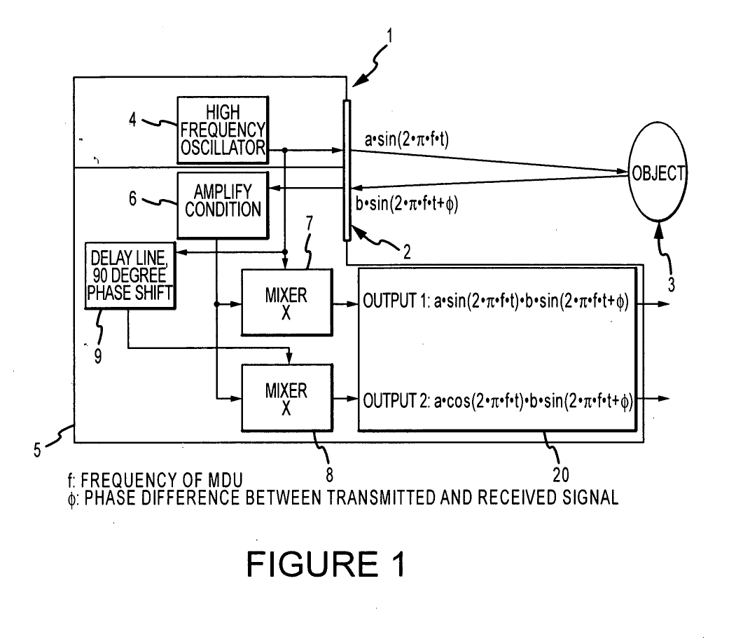

Referring to FIG. 1, a device 1 includes a transmitter emitting a signal toward an object 3 which reflects the signal and a receiver for the reflected signal. In the illustrated embodiment, the signal is a microwave signal. The expression “microwave signal” in the context of the present application refers to an electromagnetic signal which comprises at least one component in the microwave frequency range. In an embodiment of the invention the signal is a continuous wave signal as opposed to a pulse signal. The device according to the invention does not perform modulation of the transmitted or the received signals as it is based on measurement at the wavelength of the transmitted signal.

In the drawing the transmitter and the receiver are shown as a single element 2, but separate and more specialized transducers may also be used. The transmitted signal is generated by a high frequency oscillator 4, and the received signal is received from the receiving transducer by a processor 5 comprising a device 6 which amplifies and conditions the signal before transmitting it to first and a second mixers 7, 8. In the example illustrated in FIG. 1 the signal generated by oscillator 4 is a sine signal. The first mixer 7 receives signals from the oscillator 4 and from the receiver via device 6, and mixes these to provide a first signal component which in this case is a sine signal combination. The second mixer 8 is coupled to the device 6, as well as to a delay line 9. The delay line 9 performs a 90 degree phase shift to the signal transmitted from the oscillator 4 to the mixer 8 to provide a signal in quadrature with the signal transmitted from the oscillator 4. A second signal component is generated in mixer 8 which component in this case is a cosine signal combination. Processor 5 also includes an output device 20 providing an output signal representative of the patient's breathing movements based on the first and second signal components provided by mixers 7 and 8, respectively.

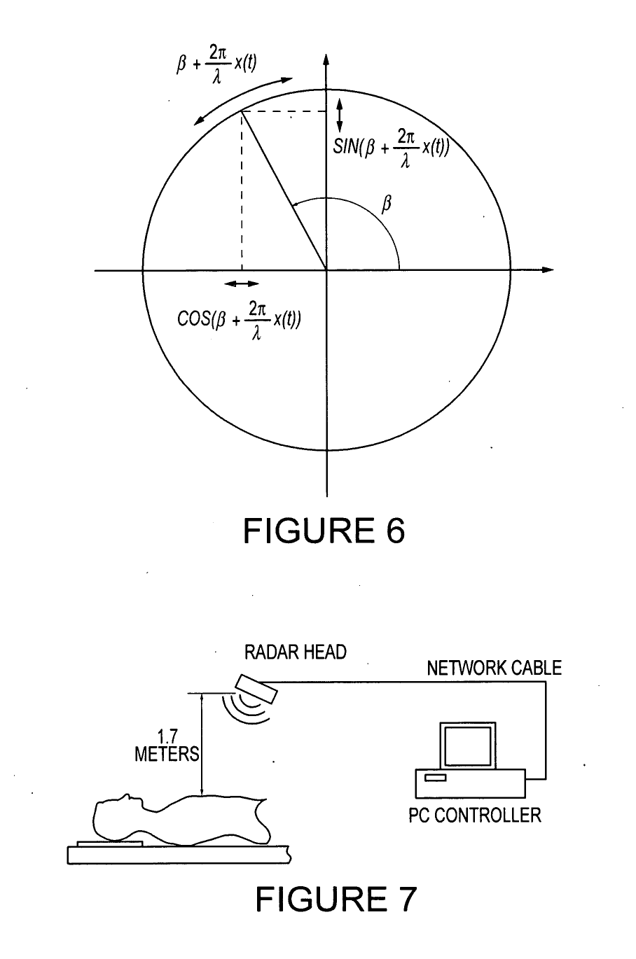

The embodiment of the invention shown in FIG. 1 will be described more in detail below by elaborating the mathematical background in a simplified form. A more exhaustive elaboration may be found below with respect to FIG. 6.

In the device of FIG. 1, the device 1 transmits a constant sine wave, a representing the amplitude of the emitted signal:

a·sin(2·π·f·t)

The signal reflected by the object and received by device 1 is:

b·sin(2·π·f·t+φ)

b represents the amplitude of the received wave, Φ represents the phase difference between transmitted and received signal in radians.

Two signal components are provided by mixers 7, 8 within device 1. Mixers 7 and 8 generate the following outputs:

a·sin(2·π·f·t)·b·sin(2·π·f·t+φ)

a·cos(2·π·f·t)·b·sin(2·π·f·t+φ)

Expanding these equations using a low pass filter with a corner frequency much less than 2*Π*f*t, the equations simplifies to:

$$\frac{1}{2} a \cdot b \cdot cos ( φ )$$

$$\frac{1}{2} a \cdot b \cdot sin ( φ )$$

These equations show that the output from output device 9 is a DC voltage for a stationary object.

If the object moves towards or away from device 1 at a constant speed, the output will be a sine/cosine wave with a frequency of 2*v*f/c, where v is the speed of the object in m/s, c is the speed of light in m/s, and f is the frequency of the microwave signal.

If an object moves in a cyclic manner around a fixed distance from device 1, the output from output device 20 will be highly dependent upon the amplitude of movement. Movement of one wavelength peak-peak or more will generate complex waveforms in the time domain with a number of harmonics of the fundamental (object cyclic frequency). If the object is at certain distances from the radar the fundamental will be of zero amplitude irrespective of the amplitude of movement of the object. The cosine output will be zero at the fundamental if the distance to the object is a full number of wavelengths of the radar operating frequency. The sine output will be zero at the fundamental if the distance to the object is a full number of wavelengths +/−⅛ of a wavelength.

This shows that a device situated at a certain distance from a patient and providing only one output (sine or cosine signal component) will in some cases fail to yield correct results in an FFT (fast Fourier transform) analysis.

The invention thus relates to the extraction of both sine and cosine signals from the receiver and demodulating both in order to obtain an unambiguous measurement of the chest movements of a patient. By keeping track of both sine and cosine signals an unambiguous representation of the movements of the patient is obtained.

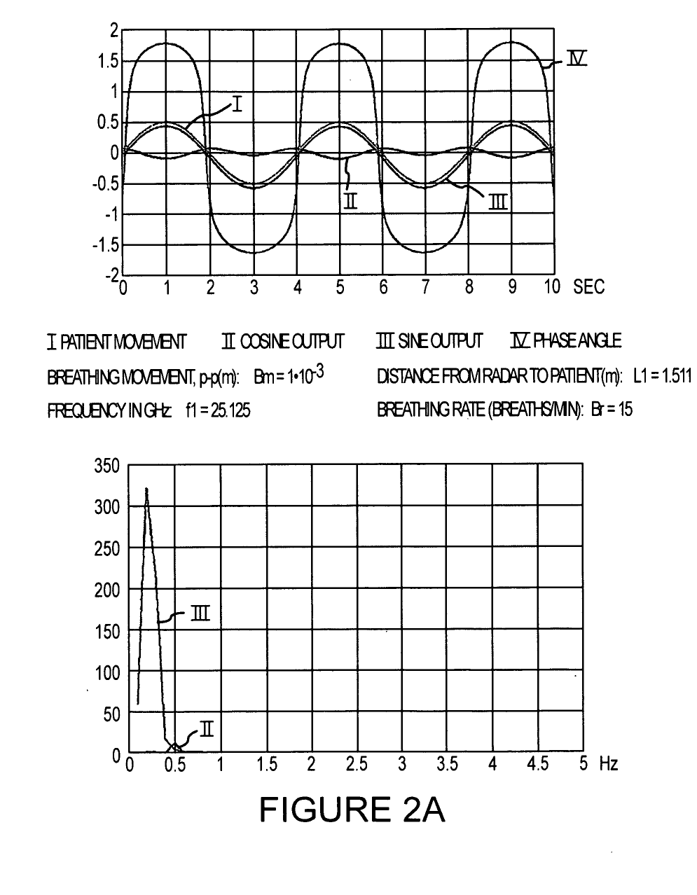

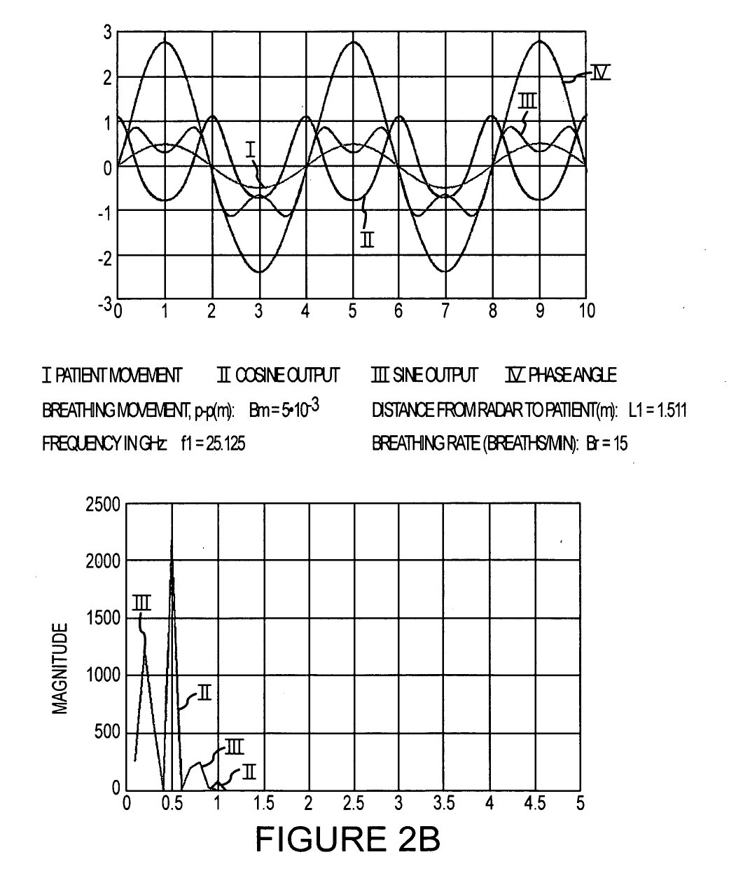

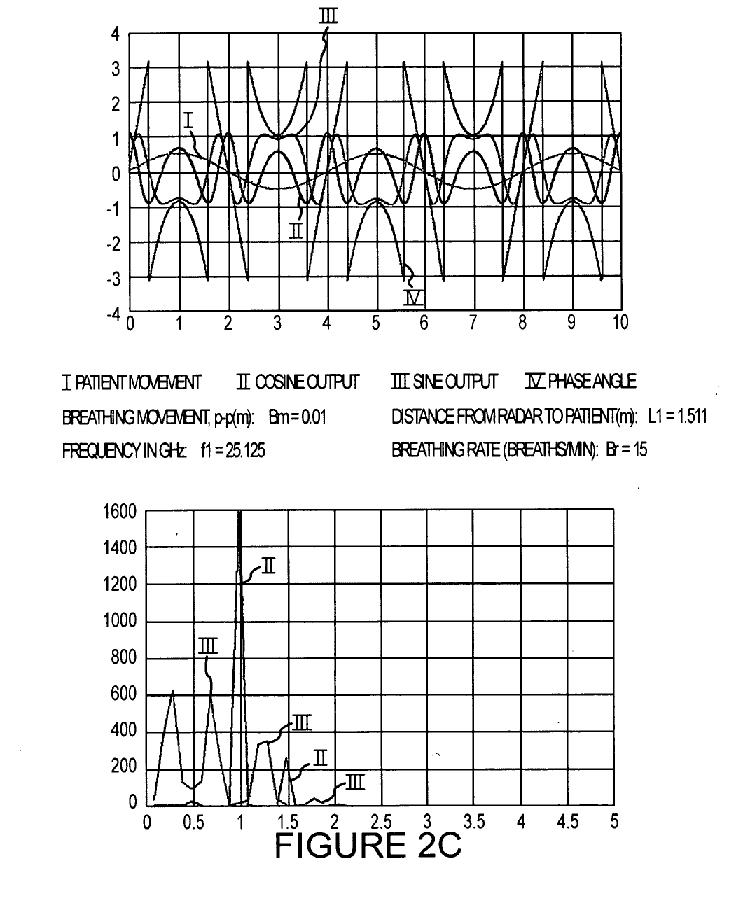

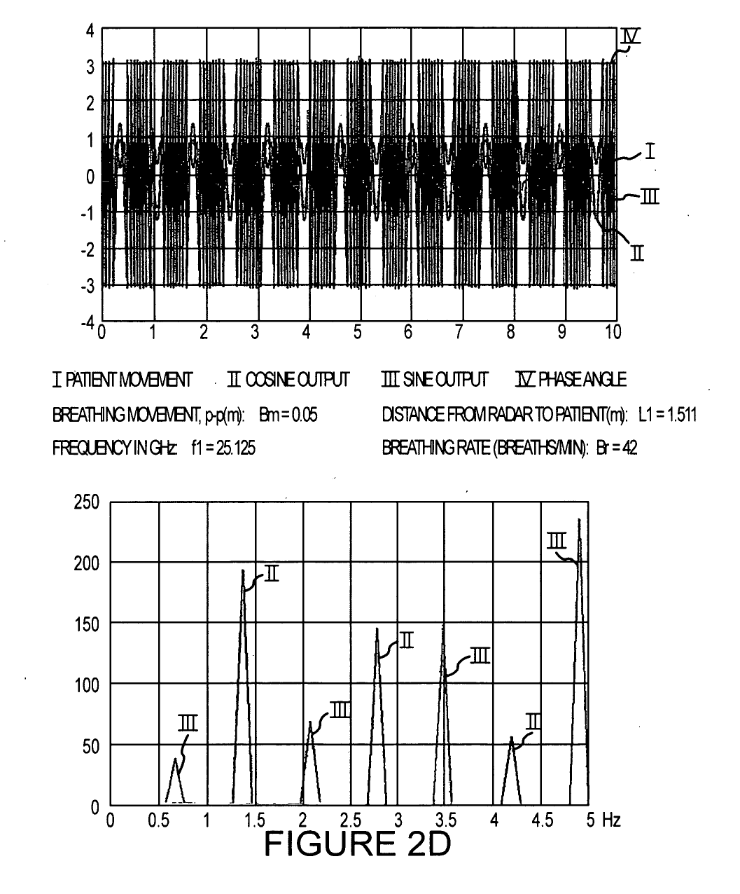

FIGS. 2A-2D show examples of output signals provided by output device 20. In the diagrams, signal I represents the patient's breathing movement. Signal II represents the second component which results from mixing a signal in quadrature with the emitted signal with the received signal in mixer 8. Signal III represents the first component which results from mixing the emitted signal with the received signal in mixer 7. Signal IV represents the angle Φ of a vector formed by the first and the second signal components. Signal IV will change in amplitude as the vector formed by the first and the second signal components arranged in quadrature rotates, representing in a certain scale the movements of the object as long as phase wrapping is compensated for.

FIG. 2A shows the above mentioned signals in a case where the chest oscillates with an amplitude of 1×10⁻³ m at a rate of 15 breaths/minute (0.25 Hz). The frequency f of the emitted signal is 25 GHz and the distance from the radar to the patient is 1.5 m. The upper part of FIG. 2A shows signals I, II, III and IV, while the lower part shows a FFT of signals II and III. As one can see on the lower part the first harmonic of signal III has a frequency in the range of the breathing frequency and thus the output of mixer 8 provides a signal representative of the breathing signal.

FIG. 2B illustrates the case where the chest movement is an oscillation with an amplitude of 5×10⁻³ m and with a rate of 15 breaths/minute (0.25 Hz). The frequency f of the emitted signal is 25 GHz and the distance from the radar to the patient is 1.5 m. In this case as in the former case the first harmonic of the first component (signal III) lies in the same frequency range as the breathing movement.

FIG. 2C illustrates the case where the chest movement is an oscillation with an amplitude of 0.01 m and with a rate of 15 breaths/minute (0.25 Hz). The frequency f of the emitted signal is 25 GHz and the distance from the radar to the patient is 1.5 m. In this case, the first harmonic of the first component also lies in the same frequency range as the breathing movement.

FIG. 2D illustrates the case where the chest movement is an oscillation with an amplitude of 0.05 m at a rate of 42 breaths/minute (0.7 Hz). The frequency f of the emitted signal is 25 GHz and the distance from the radar to the patient is 1.5 m. In this case, the first harmonic of the first component (signal III) also lies in the same frequency range as the breathing movement.

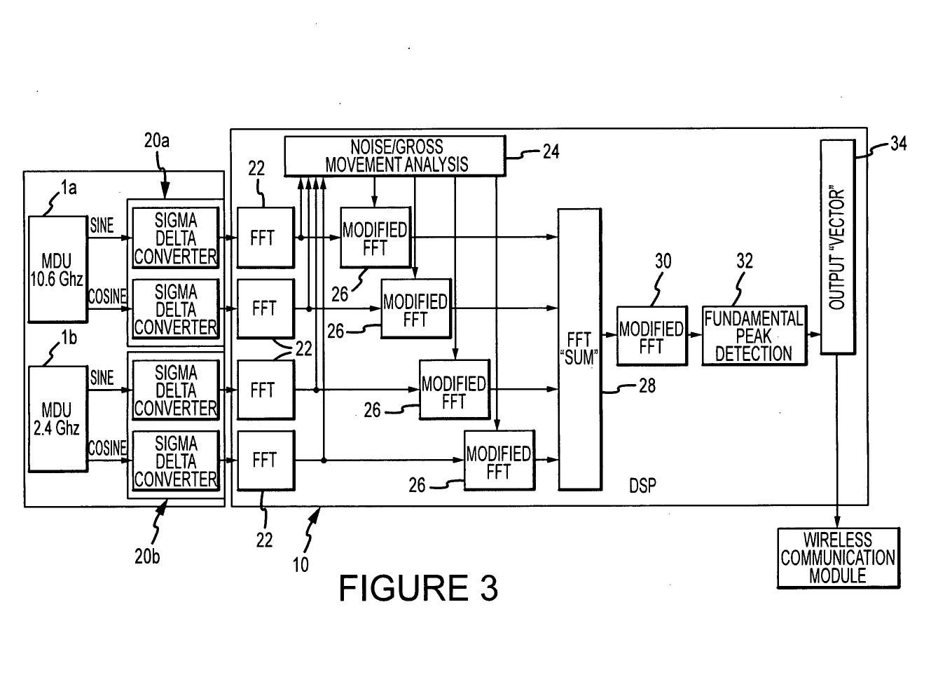

FIG. 3 illustrates an embodiment of the invention comprising generation of two microwave signals. In FIG. 3, the elements with the same function as those depicted in FIG. 1 are given the same reference numbers. In the embodiment of FIG. 3, the device 1 is replaced by two devices 1a, 1b each transmitting a different microwave signal (e.g. 10.6 GHz and 2.4 GHz) and each comprising a processor with a first mixer, a second mixer as described with respect to the device 1 of FIG. 1. Each device 1a, 1b may be coupled to a corresponding output device (20a, 20b). A third output device 10 is arranged to combine signals from the first and second output devices 20a, 20b to provide a signal representative of the patient's breathing movements. Device 10 shows a possible processing of the signals using a DSP. The signals are converted to the frequency domain by FFT circuits 22. The signals are analyzed for frequency content by “noise/gross movement analysis” circuit 24 and modified by modified FFT circuits 26 in order to minimize the effect of noise and gross movement in the breathing frequency range. The output of the FFT circuits 22 and modified FFT circuits 26 are combined by an FFTsum circuit 28 and analyzed to find the fundamental of breathing by a modified FFT circuit 30 and Fundamental peak detection circuit 32, resulting in an Output Vector 34 describing the breathing movement. Although the example shows two specific frequencies, use of three, four, or more different frequencies is comprised in the scope of the invention.

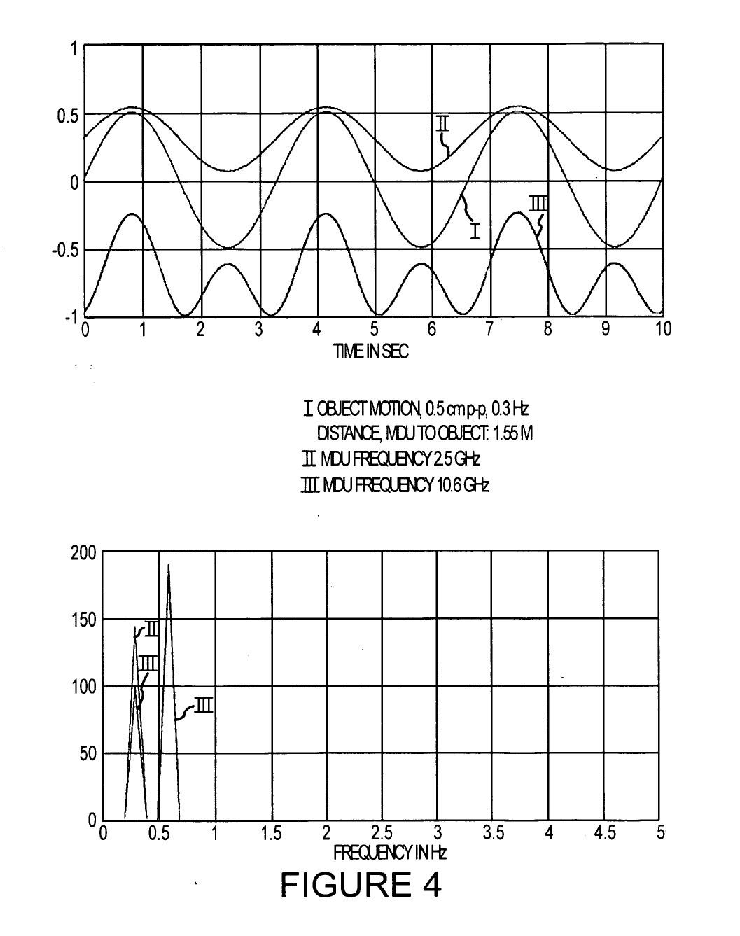

FIG. 4 shows time and frequency diagrams of signals with two different frequencies. In FIG. 4, curve I illustrates the object motion, 0.5 cm p-p (peak-peak), 0.3 Hz. Curve II shows the output of the first mixer for the device 1b having a microwave signal with a frequency of 2.5 GHz. Curve III shows the output of the first mixer for the device 1a having a microwave signal with a frequency of 10.6 GHz. FIG. 4 illustrates only the output of the first mixer in each device, that is the first component of the signal. The distance between the transducer and the object is 1.55 m.

Using two signals with different frequencies may advantageously increase the

dynamic range

$$\pm \frac{λ}{2}$$

in which the respiration signal x(t) can be uniquely decoded, since the wavelength is tied to the carrier frequency by

$$λ = 2 π \frac{v}{ω}$$

where ν v is the wave velocity. The chosen wavelengths in this case (the choices being somewhat restricted by microwave physics) are f1=2.4 GHz and f2=10.6 GHz, corresponding to wavelengths of λ₁=12.5 cm and λ₂=2.8 cm, respectively.

As mentioned above in connection with use of a single frequency, in this case the fundamental frequency of the movement (breathing) will also only dominate the spectrum if the movement is small compared to the wavelength sent by the device. The wavelength in this case being 12.5 cm at 2.5 GHz and 2.8 cm at 10.6 GHz.

Movement close to a wavelength peak to peak (p-p), will generate complex waveforms in the time domain, and a number of harmonics of the fundamental. Typically the fundamental frequency will have much less energy than higher harmonics. Movement at or around a distance to the object equal to a whole number of wavelengths, will not generate a significant output at the fundamental frequency no matter what the amplitude of the movement is. In order to cover breathing movements typical of humans, a system may advantageously use two widely spaced frequencies. The probability of a patient being at a distance of a whole number of wavelengths over any significant period is remote given the fact that a patient is a highly variable object.

As one can see, this embodiment of the invention comprising two carrier systems—each with dual demodulation using both the transmitted signal and its 90 degrees shifted version—allows for unique extraction of the respiration signal, and subsequent determination of the respiration period. This is particularly true when the respiration is confined to a peak-to-peak movement of either 12.5 cm or 2.8 cm for the two carrier systems illustrated in the example.

In addition to extracting the frequency of the movements of the object 3, other parameters, such as amplitude, may also be found by analyzing the signals further, especially if respiration rate is to be measured. Also, the analysis may comprise techniques for omitting signals related to singular movement or movements exceeding a certain amplitude, e.g. from the patient turning or shifting in bed during the measurements. In the frequency domain such movements are naturally omitted from the data.

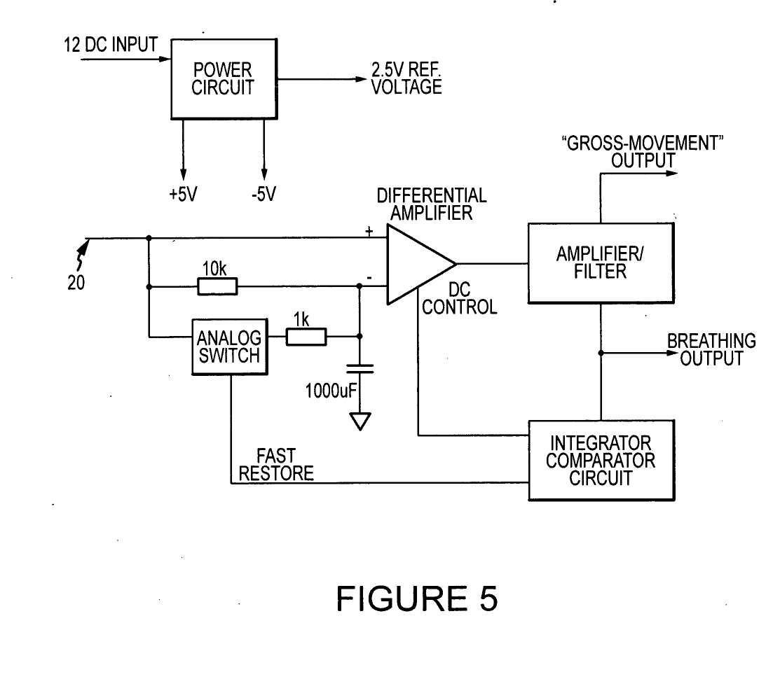

FIG. 5 shows an example of an amplifier for use in a device according to the invention. The amplifier's input is provided by the output device 20 and the amplifier comprises a low pass filter to keep only the low frequencies. The output from the amplifier can be a signal representative of a “breathing output” which signal may, for example, be shown in a monitor, activate a sound emitting device, or activate a tactile device. It can also be a “gross movement” output which can be displayed in a similar manner to indicate patient movement.

Below is a more specific description of a microwave design for successful determination of a patient's respiration rate. It is pointed out that dual demodulation may be used to provide reliable estimation of a patient's respiration rate. Furthermore, two separate carrier systems as illustrated in relation to one embodiment of the invention are beneficial for increasing the dynamic range of the measurement system. As noted above, the system may comprise a combination of microwave technology with digital signal processing.

In an example comprising demodulation of the respiration signal in only one channel the transmitted wave is given by A cos(ωt) where A denotes the amplitude and ω the angle frequency in radians. The reflected wave as seen by the receiver unit is then given by

B cos(ωt+φ(t)) (1)

where B is the (attenuated) amplitude and φ(t) is the phase delay accounted for during the transmit-reflect-receive procedure. The phase delay can be split into constant and time-variant components as follows:

$$φ ( t ) = \frac{2 π}{λ} ( l + x ( t ) ) \qquad ( 2 )$$

In Equation (2) l denotes the average distance in meters for the total transmission path, whereas x(t) is the time dependent deviation from this long term time average, i.e. the period of x(t) is the desired breathing frequency. It follows that the average value of x(t) is zero. The

$$\frac{2 π}{λ}$$

factor gives the sensitivity of the measurement system as it converts patient movement in meters into phase deviation in radians.

In the microwave frequency region (e.g. 2-18 GHz) the wavelength λ is in the 1.7-15 cm range. It follows that

$$\frac{l}{λ} >> 1$$

thus making the constant term in Equation (2) a large factor multiplied by 2π. We can therefore expand it further as:

$$φ ( t ) = 2 π n + β + \frac{2 π}{λ} x ( t ) = β + \frac{2 π}{λ} x ( t ) \qquad ( 3 )$$

where β is the non-integer part of the 2π cycles. In practice, l will be varying and the value of β is not known. A good model is to assume β to be a stochastic variable uniformly distributed between 0 and 2π. As will be pointed out in the following subsection, the value of β has significant consequences for the performance of the system if special considerations are not made. One useful form of demodulation is to multiply the received waveform with the transmitted waveform, i.e.

$$AB cos ( ω t ) cos ( ω t + φ ( t ) ) = \frac{AB}{2} [ cos ( φ ( t ) ) + cos ( 2 ω t + φ ( t ) ) ] \qquad ( 4 )$$

which after removal of the high-frequency components in the 2ω region by low-pass filtering gives us the end result:

$$c ( t ) = \frac{AB}{2} cos ( φ ( t ) ) = \frac{AB}{2} cos ( β + \frac{2 π}{λ} x ( t ) ) \qquad ( 5 )$$

Complementary information can be extracted from the received signal by separately demodulating with both sine and cosine functions. This is due to orthogonality. Following the same procedure as before, but substituting the cos(ωt) factor with sin(ωt) we get:

$$AB sin ( ω t ) cos ( ω t + φ ( t ) ) = \frac{AB}{2} [ sin ( φ ( t ) ) + sin ( 2 ω t + φ ( t ) ) ] \qquad ( 6 )$$

which after low-pass filtering gives us:

$$s ( t ) = \frac{AB}{2} sin ( φ ( t ) ) = \frac{AB}{2} sin ( β + \frac{2 π}{λ} x ( t ) ) \qquad ( 7 )$$

Note that this method may advantageously use a sine signal in the receiver, or rather a 90 degree delay of the transmitted wave. This signal may advantageously be produced using microwave technology.

FIG. 6 provides a visual interpretation of a method for measuring a patient's breathing rate. Using the two modulation techniques in the previous subsections we obtained the signals c(t) (without phase shift), and s(t) (with phase shift). Both signals may advantageously be used for reliable respiration rate measurement.

Ignoring the constant factors

$$\frac{AB}{2}$$

in Equations (5) and (7) we can illustrate how the desired information (the respiration rate) can sometimes be contained in c(t), sometimes in s(t) and sometimes in both. This is dependent on the value of β. FIG. 6 depicts the situation when β is around 120 degrees. The angle deviation from β is given by x(t) and for now we assume a low sensitivity making the deviation about 20 degrees on either side of β.

Both signals contain the desired information is this example. A spectrum analysis of both c(t) and s(t) would reveal the fundamental frequency of x(t) since the projection onto each axis preserves this.

Relying on only one signal however, can lead to a failure in the following cases:

1. β is either close to 0 or 180 degrees. The fundamental frequency of c(t) is double that of x(t).

2. β is either close to 90 or 270 degrees. The fundamental frequency of s(t) is double that of x(t).

It should be noted that using both c(t) and s(t) the respiration signal x(t) itself (not just its period) can be extracted uniquely using straightforward trigonometry. For uniqueness the movement may advantageously be confined to a region of size λ (meters).

Some embodiments disclosed thus relate to the extraction of both sine and cosine signals from the receiver and demodulating both in order to obtain an unambiguous measurement of the chest movements of a patient. By keeping track of both sine and cosine signals an unambiguous representation of the movements of the patient is obtained. In addition, two carrier frequencies may be used according to the invention to increase the dynamic range of the system.

In addition to extracting the frequency of the movements of the object, other parameters, such as amplitude, may also be found by analyzing the signals further, especially if respiration rate is to be measured. Also, the analysis may comprise techniques for omitting signals related to singular movement or movements exceeding a certain amplitude, e.g. from the patient turning or shifting in bed during the measurements. In the frequency domain such movements are naturally omitted from the data.

One embodiment of the invention, a device comprising a Doppler radar was submitted to a validation study using a resuscitation mannequin. The prototype used was a prototype breathing monitor that uses Doppler radar in constant wave mode at 24.125 GHz with a low power output in compliance with all relevant safety standards. The device was validated in a simulation study by testing the accuracy of the breathing rate recorded by the device compared to the breathing rate of a human mannequin (METI) lying supine on a standard bed and ventilated using a positive pressure ventilator (Siemens Servo 900c). Breathing rate was measured at ventilator tidal volumes (150-950 mls) that produced mannequin chest expansion ranging from barely perceptible (to an experienced clinical observer) to maximally inflated. Mannequin breathing rates were varied from 5 to 45 breaths/minute (bpm) in steps of 5 bpm. Because of limitations of the ventilator and compliance of the mannequin, large tidal volumes were not deliverable at the highest ventilation rate. Agreement between 52 pairs of mannequin and BedAlert breathing rates was assessed using a Bland-Altman plot. The mean difference between paired breathing rates (bias) was 0.899 bpm and the standard deviation of the difference (precision) was 0.873 bpm, giving limits of agreement of +2.61 to −0.812 bpm. Tidal volume made no appreciable difference to the agreement between measurements (except for the inability to drive at the highest rate). The system gives thus a clinically acceptable agreement in breathing rate with that of a ventilator driven human mannequin. Variations of tidal volume do not affect the result.

A prototype was also tested with humans, again using a constant wave Doppler radar at 24.125 GHz. Breathing rate and statistical information pertaining to the signal quality was obtained using an algorithm designed to reduce the effect of noise caused by patient movement other than breathing. The sensitivity of the system was limited to a cone of 38° by 45° degrees directly below the radar unit, thus eliminating noise caused by additional movement in the vicinity of the patient. FIG. 7 shows the arrangement of the components for the study.

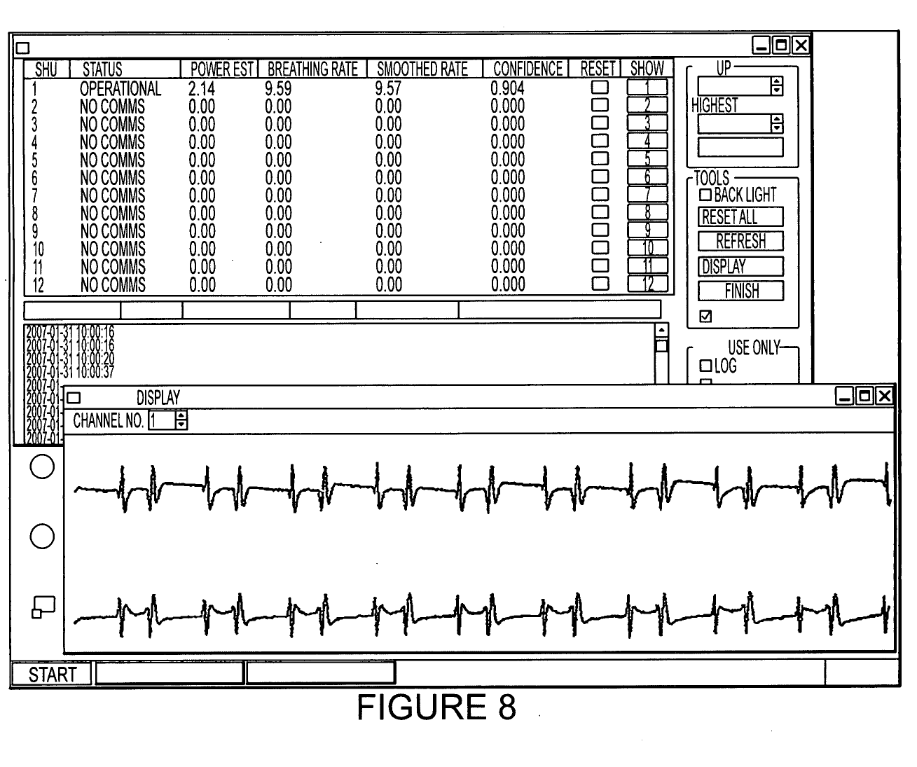

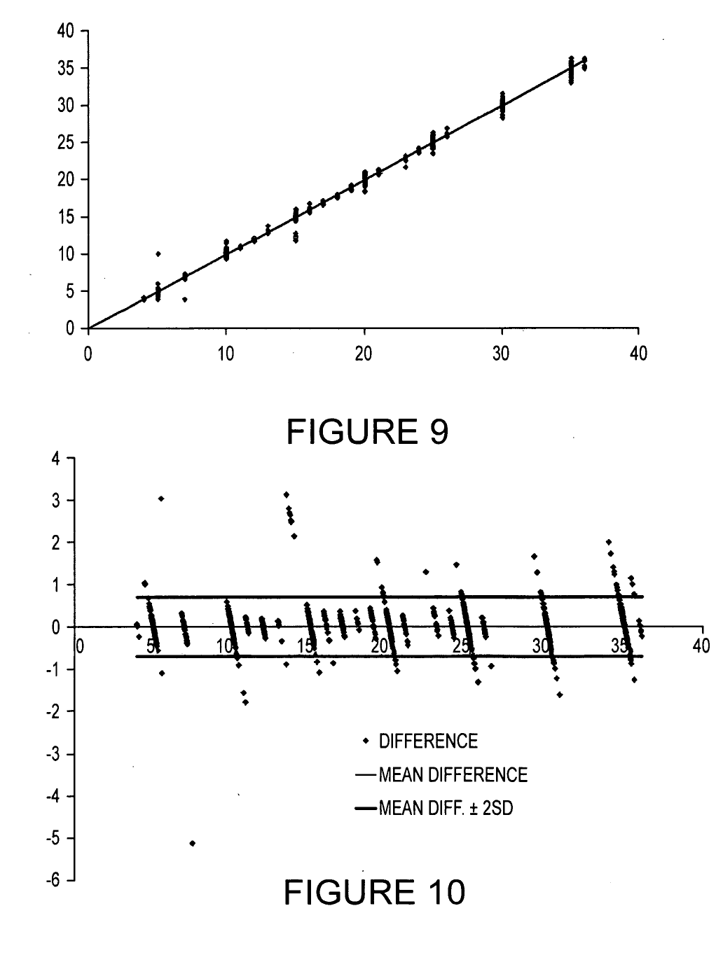

FIG. 8 illustrates a typical waveform and output from the system as displayed on the control PC. The device updates its estimate of breathing rate every 10 seconds. Its internal algorithms also provide a measure of the validity of the breathing rate estimate. Only results where the validity measure was >0.3 were accepted by the device. Six normal volunteers (3 female) took part in the study. A computer-based metronome produced a repetitive tone at a set rate, which was played to each subject via headphones. The participants were asked to begin to inspire when each tone was heard. Timing of expiration was voluntary. The tone rate was varied between 5 and 35 beats per minute in steps of 5 beats/minute. Each volunteer was studied in 4 positions: supine, prone, right lateral decubitus position and sitting in a bed at 30 degrees. Each tone rate was maintained for 2 minutes with a minute in-between for transition. Breathing rate was simultaneously recorded by the device according to the invention. A plot of the two was generated (FIG. 9). In order to assess the agreement between the two systems of measurement, a Bland Altman plot [8] was performed (FIG. 10). Agreement between 2105 pairs of tone-driven human and the device's breathing rates was assessed using a Bland-Altman plot. The mean difference between paired breathing rates (bias) was 0.010 bpm and the standard deviation of the difference (precision) was 0.348 bpm, giving limits of agreement of 0.692 to −0.672 bpm. Volunteers' position made no appreciable difference to the results. Artifacts were produced by gross movement.

The device according to the invention gives thus a clinically acceptable agreement in breathing rate with the tone-driven breathing rate of human volunteers in the physiological range of human breathing. The apparent accuracy and continuous nature of its output, suggest that the system may have potential benefits in monitoring patients.

What is claimed is:

1. A device for remote monitoring of breathing movements of a patient, comprising:
a transmitter transmitting a microwave signal toward the patient;
a receiver arranged to receive a signal reflected by the patient; and
a processor, comprising:
a first mixer arranged to mix the transmitted signal and the received signal to provide a first signal component;
a second mixer arranged to mix one of the received signal or the transmitted signal with a signal in quadrature with the other of the transmitted signal or the received signal to provide a second signal component; and
an output device arranged to provide a signal representative of the patient's breathing movements based on the first and second signal components.

2. The device of claim 1 wherein the second mixer is operable to mix the received signal with a signal in quadrature with the transmitted signal to provide the second signal component.

3. The device according to claim 1 wherein the microwave signal has a frequency in the range of 10.6 GHz-30 GHz.

4. The device of claim 3 wherein the microwave signal has a frequency in the range of 20 GHz-26 GHz.

5. The device of claim 1 wherein the microwave signal is a non-modulated signal.

6. The device of claim 1 wherein the microwave signal is a continuous wave signal.

7. The device of claim 1 wherein the output device is arranged to provide a signal resulting from the addition of the first signal component to the second signal component.

8. The device of claim 1 wherein the transmitted signal comprises two microwave signals of different frequency and the system comprises a processor with a first mixer, a second mixer and an output device for each microwave signal.

9. The device of claim 8 wherein the output device is arranged to combine signals from the first and second mixers to provide a signal representative of the patients breathing movements.

10. The device of claim 8 wherein the microwave signals have a frequency in the range of 10.6 GHz-30 GHz.

11. The device of claim 8 wherein the microwave signals have a frequency in the range of 20 GHz-26 GHz.

12. A device for remote monitoring of breathing movements of a patient, comprising:
a transmitter transmitting at least two electromagnetic signals toward the patient, the at least two electromagnetic signals having respective frequencies that are different from each other;
a receiver arranged to receive a signal reflected by the patient; and
a processor operable to mix the transmitted signal and the received signal to provide a first signal component, and to mix one of the received signal or the transmitted signal with a signal in quadrature with the other of the transmitted signal or the received signal to provide a second signal component; the processor further being operable to provide a signal representative of the patient's breathing movements based on the first and second signal components.

13. The device of claim 12 wherein the processor is operable to mix the received signal with a signal in quadrature with the transmitted signal to provide the second signal component.

14. The device of claim 12 wherein the electromagnetic signals have a frequency in the range of 10.6 GHz-30 GHz.

15. The device of claim 11 wherein the electromagnetic signals have a frequency in the range of 20 GHz-26 GHz.

16. A method for remote monitoring of breathing movements of a patient, comprising:
transmitting an electromagnetic signal toward the patient;
receiving a signal reflected by the patient;
mixing the transmitted signal and the received signal to provide a first signal component;
mixing one of the received signal or the transmitted signal with a signal in quadrature with the other of the transmitted signal or the received signal to provide a second signal component; and
combining the first and second signal components to provide a signal representative of the patient's breathing movements.

17. The method of claim 16 wherein the act of mixing one of the received signal and the transmitted signal with a signal in quadrature with the other of the transmitted signal and the received signal to provide the second signal component comprises mixing the received signal with a signal in quadrature with the transmitted signal to provide the second signal component.

18. The method of claim 16 wherein the electromagnetic signal comprises a microwave signal.

19. The method of claim 17 wherein the microwave signal has a frequency in the range of 10.6 GHz-30 GHz.

20. The method of claim 19 wherein the microwave signal has a frequency in the range of 20 GHz-26 GHz.

21. The method of claim 16 wherein the electromagnetic signal is a non-modulated signal.

22. The method of claim 16 wherein the electromagnetic signal comprises a continuous wave signal.

23. The method of claim 16 further comprising providing a signal resulting from the addition of the first signal component to the second signal component.

24. The method of claim 16 wherein the transmitted signal comprises two electromagnetic signals of different frequency, and the method further comprises:
mixing each received signal with its corresponding transmitted signal and with a signal in quadrature with the transmitted signal to provide first and second signal components; and
combining the first and second signal components to provide signals representative of the patient's breathing movements.

25. The method of claim 24, further comprising mixing signals from the first and second mixers to provide a signal representative of the patient's breathing movements.

26. A method for remote monitoring of breathing movements of a patient, comprising:
transmitting first and second microwave signals toward the patient, the first and second microwave signals having respective frequencies that are different from each other;
receiving first and second received signals from the patient from reflections of the first and second transmitted microwave signals, respectively;
mixing the first received signal with the first transmitted signal to provide a first signal component;
mixing the second received signal with the second transmitted signal to provide a second signal component;
mixing one of the first received signal or the first transmitted signal with a signal in quadrature with the other of the first transmitted signal or the first received signal to provide a third signal component;
mixing one of the second received signal or the second transmitted signal with a signal in quadrature with the other of the second transmitted signal or the second received signal to provide a fourth signal component; and
combining the first, second, third and fourth signal components to provide a signal representative of the patient's breathing movements.

27. The method of claim 26 wherein the act of mixing one of the first received signal or the first transmitted signal with a signal in quadrature with the other of the first transmitted signal or the first received signal to provide the third signal component comprises mixing the first received signal with a signal in quadrature with the first transmitted signal; and
the act of mixing one of the second received signal or the second transmitted signal with a signal in quadrature with the other of the second transmitted signal or the second received signal to provide the fourth signal component comprises mixing the second received signal with a signal in quadrature with the second transmitted signal.

28. The method of claim 26 further comprising mixing signals from the first and second mixers to provide a signal representative of the patient's breathing movements.

29. The method of claim 26 wherein the microwave signals have a frequency in the range of 10.6 GHz-30 GHz.

30. The method of claim 29 wherein the microwave signals have a frequency in the range of 20 GHz-26 GHz.
